(19) United States
(12) Patent Application Publication
(10) Pub. No.: US 2012/0271554 A1
(43) Pub. Date: Oct. 25, 2012

(54) Systems and Methods Utilizing Plethysmographic Data

(75) Inventors: Kirk H. Shelley, New Haven, CT (US); David G. Silverman, West Redding, CT (US)

(73) Assignee: YALE UNIVERSITY, New Haven, CT (US)

(21) Appl. No.: 13/322,708

(22) PCT Filed: May 28, 2010
(86) PCT No.: PCT/US10/36626

§ 371 Date: Feb. 22, 2012

Related U.S. Application Data
(60) Provisional application No. 61/182,599, filed on May 29, 2009.

(51) Int. Cl. A61B 5/0295

(52) U.S. Cl. 702/19

(57) ABSTRACT

Disclosed are apparatus, systems and methods utilizing attributes of the cardiac signal to calibrate/normalize components of the plethysmographic (PG) waveform indicating changes in venous and arterial blood volume. In the time-domain, amplitudes of respiratory-induced variations of the DC and AC components of the PG waveform may be calibrated/normalized based on an average amplitude of the PG waveform, e.g., over a respiratory cycle. Similarly, in the frequency domain, respiratory signal strength and side-band signal strength may be advantageously calibrated/normalized based on the strength of the cardiac signal or a harmonic thereof.

### CROSS-REFERENCE TO RELATED APPLICATIONS

The present application claims the benefit of co-pending provisional patent application entitled “Systems and Methods Utilizing Plethysmographic Data” that was filed on May 29, 2009 and assigned Ser. No. 61/182,599. The entire contents of the foregoing provisional application are incorporated herein by reference.

### BACKGROUND

1. Technical Field

The present disclosure relates to apparatus, systems and methods for studying and utilizing flow waveforms in the peripheral vasculature. In particular, the present disclosure relates to apparatus, systems and methods for analyzing a plethysmograph (PG) waveform, e.g., as may be obtained using a pulse oximeter.

2. Background Art

The present disclosure is related to the subject matter of U.S. Patent Publication No. 2007/0032732 to Shelley et al., entitled “Method of Assessing Blood Volume Using Photoelectric Plethysmography” (referred to herein as the “Shelley patent publication”). The Shelley patent publication is incorporated herein in its entirety.

The pulse oximeter has rapidly become one of the most commonly used patient monitoring systems both in and out of the operating room. This popularity is undoubtedly due to the pulse oximeter's ability to non-invasively monitor both arterial oxygen saturation as well as basic cardiac function (e.g., heart rhythm). In addition, a pulse oximeter is easy to use and comfortable for the patient. The present disclosure expands on the known usefulness of the pulse oximeter and pulse oximetry technology.

Pulse oximetry is a simple non-invasive method traditionally used for monitoring the percentage of hemoglobin (Hb) which is saturated with oxygen. A basic pulse oximeter includes a probe that is brought into contact with a patient, e.g., by way of attachment to a patient's finger, ear, forehead, etc., which is linked to a computerized unit for processing. A source of light originates from the probe at two wavelengths (e.g., 650 nm and 805 nm). The light is partly absorbed by hemoglobin, and the saturation level differs from wavelength-to-wavelength depending on the degree of oxygen saturation. Thus, by calculating absorption at each of the wavelengths, the processor is able to compute the percentage of hemoglobin which is oxygenated. Conventional pulse oximeter systems typically provide feedback in the form of a display indicating the percentage of Hb saturated with oxygen. Other commonly implemented informational feedback include, e.g., an audible signal for each pulse beat, a calculated heart rate, and a graphical display of changing blood volume beneath the probe.

In the process of determining oxygen saturation, a pulse oximeter inherently functions as a photoplethysmograph (PPG), measuring minute changes in the blood volume of a vascular bed (e.g., finger, ear or forehead). Thus, while the predominant application of a pulse oximeter has been calculating oxygen saturation of Hb, it is noted that the raw plethysmograph (PG) waveform is rich in information relevant to the physiology of the patient. Indeed, the PG waveform contains a complex mixture of the influences of arterial, venous, autonomic and respiratory systems on the peripheral circulation. It is important to understand, however, that the typical pulse oximeter waveform presented to the clinician is a highly filtered and processed specter of the original PG waveform. Indeed, it is normal practice for equipment manufacturers to use both auto-centering and auto-gain routines on the displayed waveforms so as to minimize variations in the displayed signal. While such signal processing may be beneficial to the determination of oxygen saturation, it often comes at the expense of valuable physiological data. Thus, due to a general lack of access to the raw PG waveform and the overriding clinical importance of monitoring oxygen saturation, various other potential uses for the PG waveform have been largely neglected.

It is disclosed in the literature that a PG waveform can be used to non-invasively measure minute changes in light absorption of living tissue. See, e.g., Hertzman, A B, “The Blood Supply of Various Skin Areas as Estimated By the Photoelectric Plethysmograph,” Am. J. Physiol. 124: 328-340 (1938). Rhythmic fluctuations in this signal are normally attributed to the cardiac pulse bringing more blood into the region being analyzed (e.g., finger, ear or forehead). This fluctuation of the PG waveform is commonly referred to as the pulsatile or AC (arterial) component. The amplitude of the AC component can be modulated by a variety of factors, including cardiac stroke volume and vascular tone. In addition to the pulsatile component of the PG waveform, there is a nonpulsatile (or weakly pulsatile) component of the PG waveform commonly referred to as the DC component. The DC component is most commonly attributed to changes in light absorption by nonpulsatile tissue, such as fat, bone, muscle and venous blood. Thus, the DC component has been correlated to changes in venous blood volume (see, e.g., paragraph [0059] of the Shelley patent publication). Apparatus, systems and methods for extracting AC and DC components of a PG waveform are provided in the Shelley patent publication.

Fluctuations in a PG waveform due to respiration/ventilation (“respiratory-induced variations”) can also be detected. See, e.g., Johansson A & Oberg P A, “Estimation of respiratory volumes from the photoplethysmographic sit. Parti: Experimental results,” Medical and Biological Engineering and Computing 37(1): 42-7 (1999). Respiratory-induced variations have been used in the past in an attempt to estimate the degree of relative blood volume of patients undergoing surgery. See, e.g., Partridge B L, “Use of pulse oximetry as a noninvasive indicator of intravascular volume status,” Journal of Clinical Monitoring 3(4): 263-8 (1987); and Shamir M, Eidelman L A et al., “Pulse oximetry plethysmographic waveform during changes in blood volume,” British Journal of Anaesthesia 82(2): 178-81 (1999).

In the Shelley patent publication, it was first noted that respiration/ventilation modulates both DC and AC components of a PG waveform. Thus, the Shelley patent publication discloses, inter alia, apparatus, systems and methods for monitoring changes in blood volume by separating the impact of respiration/ventilation on the venous and arterial systems. More particularly, by isolating the impact of respiration/ventilation on venous (DC) and arterial (AC) components of the PG waveform one is able to independently assess changes in blood volume in different regions of the vasculature (venous and arterial). As noted in the Shelley patent publication, the degree of respiratory-induced variation of the DC component of the PG waveform corresponds to venous blood volume. Similarly, as noted in the Shelley patent publication, the degree of respiratory-induced variation of the AC component of the PG waveform corresponds to arterial blood volume.

Physiologically, changes in venous blood volume often correspond to changes in end-diastolic volume (EDV), i.e., the volume of blood in the ventricles after diastole. More particularly, venous blood volume and venous compliance (e.g., relating to venous tone) affect venous blood pressure and the rate of venous return which in turn impact EDV. Thus, activation of the baroreceptor reflex, such as during acute hemorrhaging, causes venoconstriction which results in decreased venous compliance, improved venous return, and increased end-diastolic volume.

Similarly, changes in arterial blood volume correspond to cardiac stroke volume, i.e., the difference between end-systolic volume (ESV) and EDV. Cardiac output is determined as cardiac stroke volume multiplied by heart rate. Notably venous compliance is significantly (20-24 times) greater than arterial compliance.

The ability to independently monitor changes in venous and arterial blood volume has many clinical applications. For example, changes in venous and arterial blood volume may be indicative of Hypovolemia, e.g., due to bleeding, dehydration, etc. Decreased blood volume due to bleeding is, typically, characterized by an initial period of venous loss during which the cardiac output remains unaffected. With continued blood loss, decreased venous return eventually affects cardiac output (corresponding to arterial blood volume). Thus, by monitoring the degree of respiratory-induced variation of the DC component, one can detect and counter blood loss prior to cardiac output being affected. Similarly, by monitoring the degree of respiratory-induced variation of the AC component, one can detect the severity of blood loss (i.e., whether blood loss is severe enough to compromise cardiac function).

One method suggested by the Shelley patent publication for assessing changes in blood volume involves extracting DC and AC components based on the average of the PG waveform and the amplitude of the PG wavefrom, respectively. In particular, the average and amplitude may be extrapolated by comparing tracings of the peaks and valleys of the PPG waveform. The degree of respiratory-induced variation of the DC and AC components may then be monitored.

Another method suggested by the Shelley patent publication for assessing changes in blood volume involves harmonic analysis, e.g., Fourier analysis, of the PG waveform. Harmonic analysis allows for the extraction of underlying signals that contribute to a complex waveform. As disclosed in the Shelley patent publication, harmonic analysis of the PG waveform principally involves a short-time Fourier transform of the PG waveform. In particular, the PG waveform may be converted to a numeric series of data points via analog to digital conversion, wherein the PG waveform is sampled at a predetermined frequency, e.g., 50 Hz, over a given time period, e.g., 60-90 seconds. A Fourier transform may then be performed on the data set in the digital buffer (note that the sampled PG waveform may also be multiplied by a windowing function, e.g., a Hamming window, to counter spectral leakage). The resultant data may further be expanded in logarithmic fashion, e.g., to account for the overwhelming signal strength of the cardiac frequencies relative to the ventilation frequencies. It is noted that while the Shelley patent publication discloses using joint time-frequency analysis, i.e., a spectrogram, as a preferred technique for viewing and analyzing spectral density estimation of the PG waveform, a spectrum for the PG waveform, as used herein, may be extrapolated therefrom for any discrete sampling period.

According to the Shelley patent publication, PG waveform analysis, such as described above, may be used to independently monitor changes in arterial and venous blood volume. For instance, increased respiratory-induced variation of the DC component of a PG waveform, represented in the frequency domain as an increase in signal strength for the respiratory signal, is indicative of venous loss (it is noted however that decreased cardiac output may also, at times, contribute to changes in the respiratory signal). Similarly, respiratory induced variation of the AC component, represented in the frequency-domain as side-band modulation around the cardiac signal, is indicative of changes in blood volume severe enough to affect the arterial system (cardiac output). Thus, by monitoring variations in the respiratory signal, one is able to detect changes in venous blood volume. Similarly, by monitoring side-band modulation of the cardiac signal, one is able detect changes in arterial blood volume.

One of the principal challenges in analyzing the PG waveform is relatability, e.g., from patient A to patient B, ear to forehead, spontaneous respiration to positive pressure ventilation, etc. Indeed, analysis of the PG waveform, as described above, is often predicated on having a point of reference, e.g., being able to compare a sampled PG waveform relative to a “normal” PG waveform, such that changes to the PG waveform may be properly interpreted. Unfortunately, “normal” is a relative term, e.g., depending on the particular patient, measurement site, respiration state, etc. Moreover, in emergency situations, prior points of reference are not readily available. Thus, particular difficulties arise when attempting to quantify universally applicable threshold values, e.g., for instrumentation purposes.

In view of such difficulties, a need exists for improved apparatus, systems and methods for calibrating/normalizing those components of the PG waveform which are of interest. These and other needs are satisfied by the apparatus, systems and methods of the present disclosure.

### SUMMARY

Apparatus, systems and methods are provided according to the present disclosure for calibrating/normalizing components of a PG waveform which are of interest. In particular, apparatus, systems and methods are disclosed for calibrating/normalizing components of a PG waveform related to changes in venous and arterial blood volume, e.g., amplitudes of respiratory-induced variations of the DC and AC components, respectively, utilizing the cardiac signal (or a harmonic thereof). Note that calibration/normalization using the cardiac signal (or a harmonic thereof) is useful in both time-domain and frequency-domain analysis of the PG waveform. Thus, in the time-domain, amplitudes of respiratory-induced variations of the DC and AC components of the PG waveform may be calibrated/normalized based on an average amplitude of the PG waveform, e.g., over a respiratory cycle. Similarly, in the frequency domain, respiratory signal strength and side-band signal strength may be advantageously calibrated/normalized based on cardiac signal strength (or signal strength of a harmonic thereof).

Calibrated venous modulation values and scaled arterial modulation values calculated by the foregoing apparatus, systems and methods advantageously and relatably allow for detection of changes in venous blood volume and arterial blood volume, respectively. Additional features, functions and benefits of the disclosed apparatus, systems and methods will be apparent from the description which follows, particularly when read in conjunction with the appended figure.

### BRIEF DESCRIPTION OF THE DRAWINGS

To assist those of ordinary skill in the art in making and using the disclosed apparatus, systems and methods, reference is made to the appended figure, wherein:

FIG. 1 depicts extracting peaks and valleys in the time-domain from an exemplary PG waveform for determining AC and DC components thereof.

FIG. 2 depicts an exemplary PG waveform spectrum including indicators of changes in arterial and venous blood volume.

FIG. 3 depicts a spectrum of an exemplary PG waveform, wherein the respiratory signal is smaller than the first harmonic of the respiratory signal.

FIG. 4 depicts exemplary scaled AC and DC modulations, wherein the scaled AC modulation is reflective of an incorrectly determined respiratory frequency.

FIG. 5 depicts exemplary scaled AC (series 2) and DC (series 1) modulations, wherein the scaled AC modulation reflective of a correctly determined respiratory frequency.

FIG. 6 depicts a dramatic difference between the peak amplitude of a cardiac signal the integral of the cardiac signal over a range of cardiac frequencies, for an exemplary PG waveform.

### DESCRIPTION OF EXEMPLARY EMBODIMENT(S)

According to the present disclosure, advantageous apparatus, systems and methods are provided for calibrating/normalizing components of a PG waveform of the peripheral vasculature. While in exemplary embodiments, the PG waveform may be a photoplethysmograph signal (such as may be detected using a pulse oximeter, it is appreciated that any of a number of known plethymograph methods/devices may be used to detect the PG waveform. Accordingly, the present disclosure is not limited by the device used to obtain the PG waveform. Furthermore, while the present disclosure notes several exemplary measurement sites for obtaining the PG waveform (e.g., the ear, forehead, finger and esophagus), it is appreciated that any appropriate measurement site for obtaining a PG waveform of the peripheral vasculature may be used. Accordingly, the present disclosure is not limited by the measurement site used to obtain the PG waveform.

The present apparatus, systems and methods advantageously increase PG waveform relatability, e.g., between patients, measurement sites, respiration states, etc. Calibration/normalization is achieved by proportionally scaling PG waveform indicators of venous and arterial blood volume relative to the cardiac signal (or a harmonic thereof). In particular, amplitudes of respiratory-induced variations of the DC and AC components, respectively, may be scaled relative to the cardiac signal. As manifested in the spectrum of the PG waveform, the respiratory signal and the side-bands may be scaled relative to the cardiac signal (or a harmonic thereof).

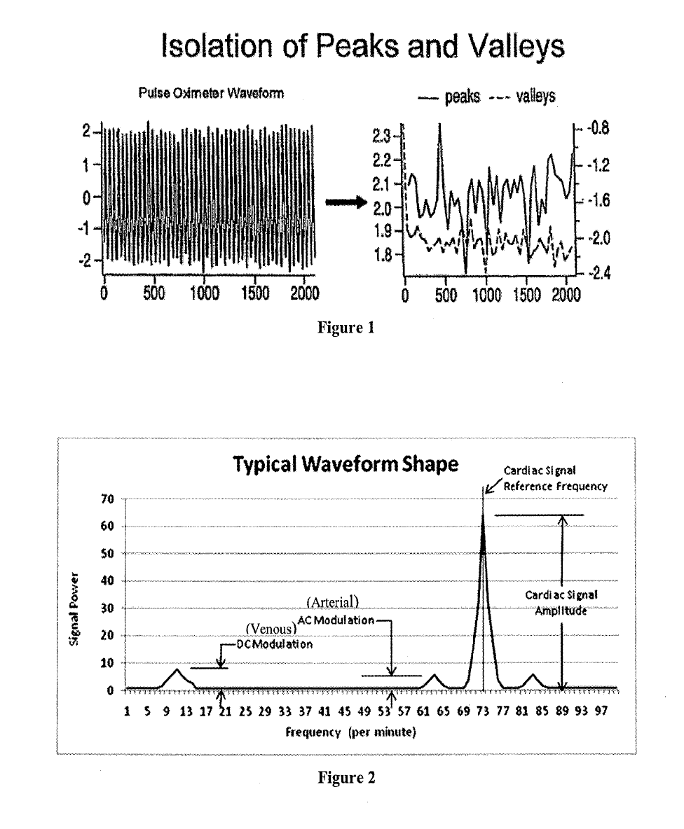

With initial reference to FIG. 1, the effects of respiration on each of the AC and DC components of the PG signal may be estimated, in the time domain, using tracings of the peaks and valleys of the PG signal. As disclosed in the Shelley patent publication, the effect of respiration on the AC component of the PG signal (also referred to herein as arterial modulation or respiratory induced variation of the AC component) may be approximated, e.g., by subtracting the tracing of the valleys from the tracing of the peaks and dividing the result by 2. Similarly, the effect of respiration on the DC component of the PG signal (also referred to herein as arterial modulation or respiratory induced variation of the AC component) may be approximated, e.g., by averaging the two tracings. The degree of respiratory-induced variation of each of the AC and DC components may be determined, e.g., over one or more respiratory cycles and calibrated/normalized relative to an average amplitude of the PG waveform, e.g., over one or more respiratory cycles.

With reference now to FIG. 2, an exemplary spectrum of a PG waveform is depicted. The exemplary PPG waveform spectrum was produced via harmonic analysis of a PG waveform from an esophageal pulse oximeter. In particular, the PG waveform was sampled at 400 Hz over a 90 second window. The spectral density (i.e., amplitude density) of the sampled PG waveform was then estimated using a fast Fourier transform (FFT). As depicted in FIG. 2, venous modulation VM (i.e., initial changes in blood volume affecting only the venous system) is reflected in the respiratory signal of PG waveform spectrum. Similarly, arterial modulation AM (i.e., subsequent changes in blood volume affecting the arterial system, e.g., affecting cardiac output) is reflected in side-bands relative to the cardiac signal.

As disclosed in the Shelley patent publication, initial increases in signal strength of the respiratory signal are usually indicative of venous loss. Thus, by monitoring shifts in the respiratory signal as manifested in a PG waveform spectrum, one is able to detect changes in venous blood volume before cardiac output is affected. Similarly, the development of side-bands around the cardiac signal, as manifested in a PG waveform spectrum, is indicative of decreased blood volume affecting the arterial system (stroke volume and cardiac output) of the subject. By monitoring changes in the side-bands, one is able to detect the degree to which cardiac function has been compromised, thus indicating the severity of blood loss. In studies conducted, such side-bands were present in all subjects experiencing decreases in blood volume greater than 300 cc.

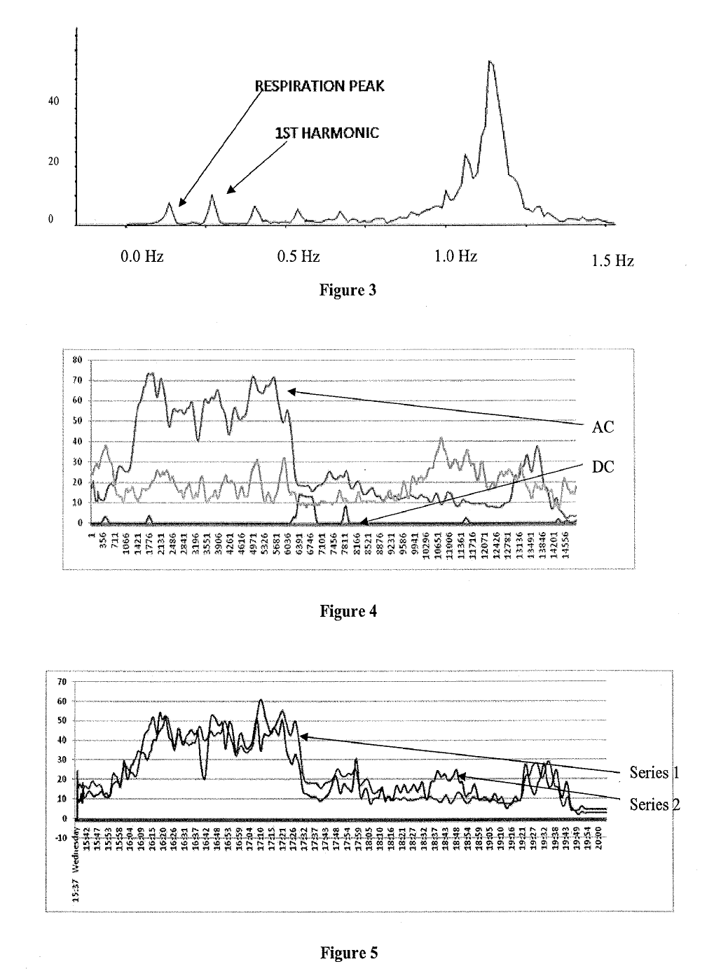

According to the present disclosure, peak detection algorithms may be advantageously applied to isolate the respiratory signal, the side-bands, and the cardiac signal (or a harmonic thereof), as manifested in a PG waveform spectrum. More particularly, a peak detection algorithm may be employed to isolate the respiratory signal by detecting the highest peak in the respiratory frequencies (e.g., 0.1-0.5 Hz). It is noted, however, that in some instances the highest peak in the respiratory frequencies may not be the respiratory signal but rather may be a harmonic thereof (see FIG. 3). Thus, if the harmonic is not properly addressed, the apparatus, systems and methods may report an incorrect respiration rate to the clinician. Furthermore, in exemplary embodiments, detecting the side-bands relies on the respiration frequency. Thus, if the respiration frequency is periodically misinterpreted, the side-band peaks would be lost (see, e.g., scaled AC modulation in FIG. 4).

Accordingly, an automatic error checking process may be implemented, e.g., to determine whether a second peak having an amplitude greater than a predetermined threshold exists at a lower frequency relative to the highest peak in the respiratory frequencies. Alternatively, an airway sensor may be used to detect an actual respiratory frequency and, thus, obviate the need for and/or supplement an error checking process (i.e., the peak closest to the actual respiratory frequency is the respiratory signal). FIG. 5 depicts scaled AC modulation (series 2) wherein a validated respiratory signal has corrected for the error depicted in FIG. 4.

A peak detection algorithm may also be employed to isolate the cardiac signal and side-bands (once the cardiac signal is identified by detecting the highest peak in the cardiac frequencies (e.g., 0.5-3 Hz), peaks on either side thereof and within the cardiac frequencies may be detected to isolate the side-bands). As disclosed in the Shelley patent publication, the spacing between the side-bands and the cardiac signal is approximately equal to the respiratory frequency.

Calibration/normalization is generally achieved by creating a ratio between the signal strengths of the feature of interest, e.g., the respiratory signal or the side-bands, relative to the signal strength of the cardiac signal (or a harmonic thereof). In exemplary embodiments, signal strength may be determined by calculating a peak amplitude for the signal. Thus, venous modulation (VM) of the PG waveform may be scaled, e.g., by dividing the peak amplitude of the respiratory signal by the peak amplitude of the cardiac signal (or a harmonic thereof). Similarly, arterial modulation (AM) of the PG waveform may be scaled, e.g., by dividing one of the peak amplitudes (or the average peak amplitude) of the side-bands by the peak amplitude of the cardiac signal (or a harmonic thereof).

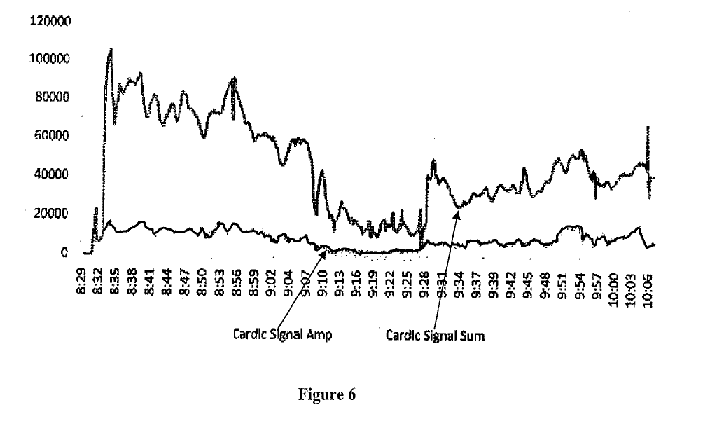

The present disclosure, however, is not limited to using peak amplitude in calculating signal strength. Indeed, other means for calculating signal strength are expressly contemplated herein. For example, signal strength may advantageously be determined over a range of frequencies characterizing a particular signal. In exemplary embodiments, the range of frequencies characterizing the signal may be determined, e.g., by noting points of inflection on either side of the peak defining the signal. Thus, signal strength may be calculated using a simple integral or root mean square of the PG waveform spectrum over the determined range of frequencies. Alternatively, a regression model may be applied to model a curve defining the signal wherein signal strength may be calculated therefrom, e.g., by computing the area under the curve. FIG. 6 depicts the dramatic difference between peak amplitude of the cardiac signal (Cardiac Signal Amp) and an integral of the cardiac signal over a range of cardiac frequencies (Cardiac Signal Sum).

In exemplary embodiments, scaled venous modulation values and scaled arterial modulation values, such as calculated by the foregoing methods, may be monitored, e.g., to detect changes in venous blood volume and arterial blood volume, respectively. Moreover, scaled venous modulation values and scaled arterial modulation values, such as calculated by the foregoing methods, advantageously provide greater relatability, e.g., between patients, measurement sites, respiration states, etc. Thus, scaled venous modulation values and scaled arterial modulation values may advantageously be compared to absolute points of reference. For instance, in exemplary embodiments, a dual warning system may be implemented, wherein an “early warning” is triggered if the scaled venous modulation value exceeds a first universally applicable threshold value (indicating venous loss) and an alarm is triggered if the scaled arterial modulation value exceeds a second universally applicable threshold value (indicating severe blood loss affecting the arterial system).

An exemplary method according to the present disclosure may generally include some combination of the following steps:

1) Sample the PG waveform, e.g., at 20 Hz over a 60 second sampling window;

2) Fast Fourier transform (FFT) the sampled PG waveform (e.g., determining amplitude density);

3) Isolate the cardiac signal (or a harmonic thereof) within the cardiac frequencies;

4) Isolate the side-bands relative to the cardiac signal;

5) Isolate the respiratory signal within the respiratory frequencies;

6) Calculate a signal strength for each of the cardiac signal (or a harmonic thereof), the side-bands, and the respiratory signal;

7) Calculate a scaled venous modulation value by dividing the signal strength of the respiratory signal by the signal strength of the cardiac signal (or a harmonic thereof);

8) Calculate a scaled arterial modulation value by dividing the signal strength of the side-bands by the signal strength of the cardiac signal (or a harmonic thereof);

10) Display the scaled venous modulation value and scaled arterial modulation value and

11) Shift the sampling window forward, e.g., by ten seconds.

Systems according to the present disclosure advantageously include a plethysmograph device (for detecting the PG waveform), e.g., a pulse oximeter, coupled with a computer or processor (for carrying out the above method). Indeed, it is explicitly contemplated that the above process of calibration/normalization of blood volume indicators in a PG waveform may be carried out, e.g., via a processing unit having appropriate software, firmware and/or hardware. As previously noted, a plethysmograph device may be used to obtain the PG waveform of the peripheral vasculature. Thus, in exemplary embodiments, the plethysmograph device may include an interface for communicating with an external processing unit. The external processing unit may, for example, be a computer or other stand alone device having processing capabilities. Thus, in exemplary embodiments, the external processing unit may be a multifunction unit, e.g., with the ability to communicate with and process data for a plurality of measurement devices. Alternatively the plethysmograph device may include an internal or otherwise dedicated processing unit, typically a microprocessor or suitable logic circuitry. A plurality of processing units may, likewise, be employed. Thus, in exemplary embodiments, both dedicated and external processing units may be used.

The processing unit(s) of the present disclosure, generally, include means, e.g., hardware, firmware or software, for carrying out the above process of calibration/normalization. In exemplary embodiments, the hardware, firmware and/or software may be provided, e.g., as upgrade module(s) for use in conjunction with existing plethysmograph devices/processing units. Software/firmware may, e.g., advantageously include processable instructions, i.e. computer readable instructions, on a suitable storage medium for carrying out the above process. Similarly, hardware may, e.g., include components and/or logic circuitry for carrying out the above process.

A display and/or other feedback means may also be included to convey detected/processed data. Thus, in exemplary embodiments, normalized values computed using the above process of calibration/normalization, e.g., scaled venous modulation values and scaled arterial modulation values, and or other PG related data may be displayed, e.g., on a monitor. The display and or other feedback means may be stand-alone or may be included as one or more components/modules of the processing unit(s) and/or plethysmograph device.

In general, it will be apparent to one of ordinary skill in the art that various embodiments described herein may be implemented in, or in association with, many different embodiments of software, firmware and/or hardware. The actual software code or specialized control hardware used to implement some of the present embodiments is not intended to limit the scope of the embodiments. For example, certain aspects of the embodiments described herein may be implemented in computer software using any suitable computer software language type such as, for example, C or C++ using, for example, conventional or object-oriented techniques. Such software may be stored on any type of suitable computer-readable medium or media such as, for example, a magnetic or optical storage medium. Thus, the operation and behavior of the embodiments may be described without specific reference to the actual software code or specialized hardware components. The absence of such specific references is feasible because it is clearly understood that artisans of ordinary skill would be able to design software and control hardware to implement the various embodiments based on the description herein with only a reasonable effort and without undue experimentation.

Moreover, the methods of the present disclosure may be executed by, or in operative association with, programmable equipment, such as computers and computer systems. Software that cause programmable equipment to execute the methods may be stored in any storage device, such as, for example, a computer system (non-volatile) memory, an optical disk, magnetic tape, or magnetic disk. Furthermore, the processes may be programmed when the computer system is manufactured or via a computer-readable medium. Such a medium may include any of the forms listed above with respect to storage devices.

It can also be appreciated that certain steps described herein may be performed using instructions stored on a computer-readable medium or media that direct a computer system to perform said steps. A computer-readable medium may include, for example, memory devices such as diskettes, compact discs of both read-only and read/write varieties, optical disk drives and hard disk drives. A computer-readable medium may also include memory storage that may be physical, virtual, permanent, temporary, semi-permanent and/or semi-temporary.

A “processor,” “processing unit,” “computer” or “computer system” may be, for example, a wireless or wireline variety of a microcomputer, minicomputer, server, mainframe, laptop, personal data assistant (PDA), wireless e-mail device (e.g., “BlackBerry” trade-designated devices), cellular phone, pager, processor, fax machine, scanner, or any other programmable device configured to transmit and receive data over a network. Computer systems disclosed herein may include memory for storing certain software applications used in obtaining, processing and communicating data. It can be appreciated that such memory may be internal or external to the disclosed embodiments. The memory may also include any means for storing software, including a hard disk, an optical disk, floppy disk, ROM (read only memory), RAM (random access memory), PROM (programmable ROM), EEPROM (electrically erasable PROM) and other computer-readable media.

Although the present disclosure has been described with reference to exemplary embodiments and implementations thereof, the disclosed systems, and methods are not limited to such exemplary embodiments/implementations. Rather, as will be readily apparent to persons skilled in the art from the description provided herein, the disclosed apparatus, systems and methods are susceptible to modifications, alterations and enhancements without departing from the spirit or scope of the present disclosure. Accordingly, the present disclosure expressly encompasses such modification, alterations and enhancements within the scope hereof.

1. A method for facilitating detection of changes in blood volume, said method comprising steps of:
sampling a plethysmograph (PG) waveform;
calculating a spectrum for the PG waveform having a cardiac signal and at least one blood volume indicator;
detecting the cardiac signal or a harmonic thereof within normal cardiac frequencies of the PG waveform spectrum;
detecting the blood volume indicator;
determining a signal strength for each of the cardiac signal or the harmonic thereof and the blood volume indicator, wherein at least one of the determined signal strengths is calculated over a range of characteristic frequencies;
calculating a normalized value for the blood volume indicator by dividing the signal strength of the blood volume indicator by the signal strength of the cardiac signal or the harmonic thereof.

2. The method of claim 1, wherein the blood volume indicator is one of: (i) a side-band detected on either side of the cardiac signal and (ii) a respiratory signal detected within normal respiratory frequencies of the PG waveform spectrum.

3. The method of claim 2, wherein the calculating a normalized value for the blood volume indicator includes either (i) calculating a scaled venous modulation value by dividing the signal strength of the respiratory signal by the signal strength of the cardiac signal or the harmonic thereof or (ii) calculating a scaled arterial modulation value by dividing the signal strength of the side-band by the signal strength of the cardiac signal or the harmonic thereof.

4. (canceled)

5. (canceled)

6. The method of claim 2, wherein the detecting the cardiac signal includes using a peak detection algorithm to detect the highest peak in the normal cardiac frequencies of the PG waveform spectrum, wherein the detecting the side-band includes using a peak detection algorithm to detect a peak on either side of the cardiac signal, wherein the spacing between the side-band and the cardiac signal is approximately equal to a respiratory frequency and wherein the detecting the respiratory signal includes using a peak detection algorithm to detect the highest peak in the normal respiratory frequencies of the PG waveform spectrum.

7. (canceled)

8. (canceled)

9. The method of claim 2, wherein the detecting the respiratory signal further includes an error checking process for checking whether a highest peak in the normal respiratory frequencies of the PG waveform spectrum is a harmonic of a true respiratory signal.

10. The method of claim 2, wherein an actual respiratory frequency is determined and the respiratory signal is detected based on said actual respiratory frequency.

11. The method of claim 1, wherein the range of characteristic frequencies are determined by points of inflection on either side of a peak defining the cardiac signal, the harmonic of the cardiac signal or the blood volume indicator.

12. The method of claim 1, wherein the at least one of the determined signal strengths is calculated as one of (i) an integral of the PG waveform spectrum over the range of characteristic frequencies and (ii) the root mean square of the PG waveform spectrum over the range of characteristic frequencies.

13. (canceled)

14. The method of claim 3, further comprising detecting changes in venous blood volume and arterial blood volume by monitoring changes in the scaled venous modulation value and scaled arterial modulation value over time.

15. (canceled)

16. The method of claim 3, further comprising comparing the scaled venous modulation value and scaled arterial modulation value to absolute points of reference, wherein the absolute points of reference are universally applicable threshold values indicative of initial blood loss affecting venous return and severe blood loss affecting cardiac output.

17. (canceled)

18. The system according to claim 32, said processing unit further including:
means for calculating a spectrum for the PG waveform having a cardiac signal and at least one blood volume indicator;
means for detecting the cardiac signal or a harmonic thereof within normal cardiac frequencies of the PG waveform spectrum;
means for determining a signal strength for each of the cardiac signal or the harmonic thereof and the blood volume indicator, wherein at least one of the determined signal strengths is calculated over a range of characteristic frequencies, wherein the signal strength for the cardiac signal or the harmonic thereof represents the average amplitude of the PG waveform and wherein the signal strength for the blood volume indicator represents the amplitude of the blood volume indicator.

19. (canceled)

20. (canceled)

21. The system of claim 18, further comprising means for comparing the normalized value for the blood volume indicator to an absolute point of reference.

22. (canceled)

23. (canceled)

24. (canceled)

25. (canceled)

26. The system of claim 18, wherein the range of characteristic frequencies are determined by points of inflection on either side of a peak defining the cardiac signal, the harmonic of the cardiac signal or the blood volume indicator.

27. The system of claim 18, wherein the at least one of the determined signal strengths is calculated as one of (i) an integral of the PG waveform spectrum over the range of characteristic frequencies and (ii) the root mean square of the PG waveform spectrum over the range of characteristic frequencies.

28. (canceled)

29. A method for facilitating detection of changes in blood volume, said method comprising steps of:
sampling a plethysmograph (PG) waveform;
detecting an average amplitude of the PG waveform over a period of time;
detecting a blood volume indicator;
calculating a normalized value for the blood volume indicator by dividing the amplitude of the blood volume indicator by the average amplitude of the PG waveform.

30. The method of claim 29, wherein the blood volume indicator is one of: (i) respiratory-induced variation of a DC component of the PG waveform and (ii) respiratory-induced variations of an AC component of the PG waveform.

31. The method of claim 29, wherein the period of time is a respiratory cycle.

32. A system for facilitating detecting changes in blood volume, said system comprising a plethysmorgraphic device coupled with a processing unit said processing unit further including:
means for sampling a plethysmograph (PG) waveform;
means for detecting an average amplitude of the PG waveform over a period of time;
means for detecting a blood volume indicator;
means for calculating a normalized value for the blood volume indicator by dividing the amplitude of the blood volume indicator by the average amplitude of the PG waveform.

33. The system of claim 32, wherein the blood volume indicator is one of: (i) respiratory-induced variation of a DC component of the PG waveform and (ii) respiratory-induced variations of an AC component of the PG waveform.

34. The system of claim 32, wherein the period of time is a respiratory cycle.
